(19) United States
(12) Patent Application Publication
(10) Pub. No.: US 2026/0183505 A1
(43) Pub. Date: Jul. 2, 2026

(54) NEONATAL INCUBATOR AND HUMIDIFIER SYSTEM THEREFOR

(71) Applicant: GE Precision Healthcare LLC, Waukesha, WI (US)

(72) Inventors: Ravi Kumar Dhulipalla, Bangalore (IN); Rajendra Naik, Bengaluru (IN)

(73) Assignee: GE Precision Healthcare LLC, Waukesha, WI (US)

(21) Appl. No.: 19/002,946

(22) Filed: Dec. 27, 2024

(51) Int. Cl. A61M 16/16; A61G 11/00; A61M 16/10

(52) U.S. Cl. CPC A61M 16/16; A61G 11/00; A61M 16/109; A61M 2205/3368; A61M 2205/3375; A61M 2205/3389; A61M 2240/00

(57) ABSTRACT

A humidifier system for humidifying a microenvironment in a neonatal incubator includes a reservoir configured to hold water to be evaporated for humidifying the microenvironment and a plurality of heating elements, each heating element positioned at a different height between a top side and a bottom side of the reservoir. A controller is configured to control power to each of the plurality of heating elements to heat a surface region of the water to a higher temperature than a bottom region of the water closer to the bottom side of the reservoir.

### BACKGROUND

The present disclosure generally relates to incubator systems providing a microenvironment for a neonate, and more specifically to a neonatal incubator having a humidifier system configured to reduce sound levels generated due to heating the evaporant.

Neonatal incubators create a microenvironment that is thermally neutral where a neonate can develop. These incubators typically include a humidifier and associated control system that controls the humidity in the neonatal microenvironment. The humidifier comprises a device that evaporates an evaporant, such as distilled water, to increase relative humidity of air within the neonatal microenvironment. Such humidifiers typically have an evaporant source in the form of a reservoir that holds water to be dispersed into the microenvironment within the incubator. For example, the humidifier may be a steam humidifier or vaporizer in which the water is heated to cause evaporation. The humidifier is typically controllable such that the amount of water, or water vapor, added to the microenvironment is adjustable to control the humidity to a desired value.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect of the present disclosure, a humidifier system for humidifying a microenvironment in a neonatal incubator includes a reservoir configured to hold water to be evaporated for humidifying the microenvironment and a plurality of heating elements, each heating element positioned at a different height between a top side and a bottom side of the reservoir. A controller is configured to control power to each of the plurality of heating elements to heat a surface region of the water to a higher temperature than a bottom region of the water closer to the bottom side of the reservoir.

In one embodiment, the controller is further configured to turn off power to individual heating elements of the plurality of heating elements as a water level of the water decreases.

In another embodiment, the plurality of heating elements includes at least a first heating element and a second heating element, wherein the first heating element is above the second heating element in the reservoir. The controller is further configured to determine that the first heating element is above a water level of the water in the reservoir and turn off power to the first heating element and increase power to the second heating element.

In another embodiment, the system further comprises a water level sensor configured to measure a water level of the water in the reservoir, and wherein the controller is configured to control the plurality of heating elements based on the measured water level.

In another embodiment, the system further comprises a plurality of temperature sensors, wherein each temperature sensor is configured to measure a temperature of a respective one of the plurality of heating elements, and wherein the controller is configured to control the plurality of heating elements based on the measured temperatures of the plurality of heating elements.

In another embodiment, the controller is further configured to provide more power to a top heating element than the other heating elements in the plurality of heating elements, wherein the top heating element is the heating element out of the plurality of heating elements that is at or immediately below a water level of the water in the reservoir.

In another embodiment, the controller is configured to identify the top heating element out of the plurality of heating elements, wherein the top heating element is the highest heating element that is below a water level of the water in the reservoir and provide a maximum power amount to the top heating element. Optionally, the controller is configured to provide a lesser power amount to each of a subset of heating elements below the top heating element, wherein the lesser power amount is less than the maximum power amount.

In another embodiment, the controller is configured to identify a new top heating element out of the plurality of heating elements as the water level decreases such that the new top heating element remains within surface region.

In another embodiment, the plurality of heating elements are in a floating heater system configured to float such that at least the top heating element is maintained within the surface region and at least one lower heating element is maintained below the surface region. The controller is configured to provide a maximum power amount to the top heating element and provide a lesser power amount to each of the at least one lower heating element, wherein the lesser power amount is less than the maximum power amount such that the water in the surface region is heated more than the water below the surface region.

In another embodiment, the at least one ultrasonic vibrator positioned in the surface region and configured to aerosolize the water in the surface region that is heated by the at least one heating element.

In another embodiment, the system further comprises a plurality of ultrasonic vibrators each positioned at a different height within the reservoir. The controller is further configured to activate only a top vibrator out of the plurality of ultrasonic vibrators based on a water level of the water in the reservoir, wherein the top vibrator is at or immediately below the water level.

In another embodiment, the controller is configured to control the plurality of heating elements to maintain the surface region of the water within a predetermined temperature range that is below boiling.

In another embodiment, the at least one ultrasonic vibrator is in a floating element configured to maintain the at least one ultrasonic vibrator in the surface region of the water as the water level changes.

In another aspect of the present disclosure, a neonatal incubator includes a bed configured to support a neonate, a hood above the bed configured to encapsulate a microenvironment around the neonate, a humidity sensor configured to measure the humidity of the microenvironment, and a humidifier system configured to humidify the microenvironment. The humidifier system includes a reservoir configured to hold water to be evaporated for humidifying the microenvironment, the reservoir having a top side and a bottom side, a plurality of heating elements, each heating element positioned at a different height within the reservoir, and a controller configured to control power to each of the plurality of heating elements based on a water level of the water in the reservoir and the measured humidity so as to heat a surface region of the water to a higher temperature than a bottom region of the water closer to the bottom side of the reservoir.

In one embodiment, the plurality of heating elements includes at least a first heating element and a second heating element, wherein the first heating element is above the second heating element in the reservoir. The controller is further configured to determine that the first heating element is above the water level and turn off power to the first heating element and increase power to the second heating element.

In another embodiment, the humidifier system further comprises a plurality of temperature sensors, wherein each temperature sensor is configured to measure a temperature of a respective one of the plurality of heating elements, and wherein the controller is configured to control the plurality of heating elements based on the measured temperatures of the plurality of heating elements.

In another embodiment, the plurality of heating elements are in a floating heater system configured to float in the reservoir such that at least a top heating element is maintained within the surface region and at least one lower heating element is maintained below the surface region. The controller is configured to provide a maximum power amount to the top heating element and provide a lesser power amount to each of the at least one lower heating element, wherein the lesser power amount is less than the maximum power amount such that the water in the surface region is heated more than the water below the surface region.

In another embodiment, the controller is configured to identify a top heating element out of the plurality of heating elements, wherein the top heating element is the highest heating element that is below a water level of the water in the reservoir. A maximum power amount is provided to the top heating element and a lesser power amount is provided to each of a subset of heating elements below the top heating element, wherein the lesser power amount is less than the maximum power amount.

In another embodiment, the system further comprises a plurality of ultrasonic vibrators each positioned at a different height within the reservoir. The controller is further configured to control power to the plurality of heating elements to maintain the surface region of the water within a predetermined temperature range that is below boiling and activate only a top vibrator out of the plurality of ultrasonic vibrators based on the water level, wherein the top vibrator is at or immediately below the water level.

In another aspect of the present disclosure, a method of controlling a humidifier system in a neonatal incubator is provided. The humidifier system includes a reservoir configured to hold water to be evaporated for humidifying the microenvironment and a plurality of heating elements, each heating element positioned at a different height between a top side and a bottom side of the reservoir. The method includes identifying a top heating element out of the plurality of heating elements, wherein the top heating element is the highest heating element that is below a water level of the water in the reservoir. A maximum power amount is provided to the top heating element and a lesser power amount is provided to each of a subset of heating elements below the top heating element, wherein the lesser power amount is less than the maximum power amount.

In one embodiment, the lesser power amount is zero power such that the subset of heating elements is turned off.

In another embodiment, the method further includes measuring a temperature of at least a subset of the plurality of heating elements, determining which of the at least the subset of heating elements exceeds a threshold temperature, and identifying the top heating element based on which of the at least the subset of heating elements exceeds the threshold temperature.

In another embodiment, the plurality of heating elements includes at least a first heating element and a second heating element. The method further includes determining that the first heating element is above the water level and turning off power to the first heating element and increasing power to the second heating element. Optionally, determining that the first heating element is above the water level is based on the top heating element being that the top heating elements exceeds a threshold temperature.

Various other features, objects, and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a perspective view of an exemplary incubator containing a humidifier system, including a schematic diagram depicting relevant sensing and control elements for the humidifier system.

FIG. 2 is a schematic depiction of a reservoir in an incubator system having an exemplary heating element configuration according to one embodiment of the present disclosure.

FIG. 3 is a schematic depiction of a reservoir in an incubator system having an exemplary floating heater system with a plurality of heating elements.

FIG. 4 depicts one embodiment of a humidifier system with stationary heating elements and a plurality of temperature sensors configured to sense the temperature of the plurality of heating elements.

FIGS. 5 and 6 depict methods for controlling a humidifier system of a neonatal incubator, or portions of such methods.

FIG. 7 depicts an exemplary humidifier system comprising a plurality of ultrasonic vibrators with a plurality of heaters positioned at different heights within the reservoir.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Through extensive experimentation and research in the field of neonatal incubators, the present inventors have recognized that incubator systems using boiled water to generate humidity create too much noise. Namely, the bubbles generated from boiling the water in the incubator reservoir tends to cause noise at levels that are disruptive to the neonate housed in the incubator. Where subsurface water is heated to boiling temperatures that are higher than the surface temperature, the rising vapor bubbles tend to cool due to the lower temperature of the surrounding water towards the surface. This cooling causes the bubbles to condense and collapse, causing noise.

The noise levels generated by humidifier systems negatively impact infants housed in incubators. The inventors have recognized that the sound level in an incubator can be significantly reduced by changing existing humidifier systems to reduce or eliminate the noises caused by bubbling, or boiling, the water. Present incubator systems heat the water at the bottom of the reservoir, causing the water to boil up to the top. This boiling generates a bubbling sound, which sometimes can be quite loud and the greatest source of noise generation in the incubator.

The disclosed system significantly reduces or eliminates the sound caused by the boiling water by heating the surface region of the water to a hotter temperature than the lower regions within the reservoir, and/or maintaining the water in the lower regions at a temperature below boiling so that bubbles do not form, thereby eliminating the creation of rising bubbles and eliminating the bubbling sound. The disclosed humidifier system implements a plurality of heating elements positioned at different heights within the reservoir. A controller is configured to control power to each of the plurality of heating elements to heat a surface region of the water to a higher temperature than a bottom region of the water closer to the bottom side of the reservoir. The plurality of heating elements may be powered differently based on the water level of the water in the reservoir such that the heating output moves downward as the water level in the reservoir decreases.

In one embodiment, the heating elements are stationary. Heating elements that are above the water surface are turned off, i.e., not powered, and the heating element(s) that is(are) just below the water surface are powered the most so that the surface region of the water is hotter than the bottom region of the water towards the bottom side of the reservoir. Thus, the controller is configured to identify a top heating element out of the plurality of heating elements, wherein the top heating element is the highest heating element (i.e., closest to the top side of the reservoir) that is below the water level of the water in the reservoir. A maximum power amount is provided to the top heating element such that it generates more heat than other heating elements in the heating system. A lesser power amount is provided to each of a subset of heating elements below the top heating element, wherein the lesser power amount is less than the maximum power amount. Heating elements above the top heating element are turned off or otherwise limited so that they do not overheat since they are not in the water. For example, where the plurality of heating elements includes at least a first heating element and a second heating element and the first heating element is above the second heating element in the reservoir, the controller is further configured to determine that the first heating element is above a water level of the water in the reservoir turn off power to the first heating element and increase power to the second heating element such that the heating element just below the water surface puts out the most heat.

In other embodiments, the plurality of heating elements may be in a floating heater system wherein the configured to float in the reservoir such that it moves downward in the reservoir as the water level decreases. The floating heater is configured such that a top heating element is maintained within the surface region and at least one lower heating element is maintained below the surface region, and the heating elements move downward in parallel as the water level decreases. In such an embodiment, the controller is configured to provide a maximum power amount to the top heating element that is just below the water surface and provide a lesser power amount to each of the at least one lower heating element such that the water in the surface region is heated more than the water below the surface region. In some embodiments, a low water sensor for the reservoir may be configured to detect when the floating heater system has reached the bottom side of the reservoir.

In some embodiments, the heating system includes at least one ultrasonic vibrator positioned in the surface region and configured to aerosolize the water in the surface region that is heated by the at least one of the plurality of heating elements. Where an ultrasonic vibrator is utilized to aerosolize the water, the system may be configured to control the plurality of heaters to maintain the surface region of the water within a predetermined temperature range that is below boiling, such as just below boiling, so that bubbles are not formed. In some embodiments, the ultrasonic vibrator may be in a floating element configured to maintain the at least one ultrasonic vibrator in the surface region of the water as the water level changes. In other embodiments, the system may include a plurality of ultrasonic vibrators each positioned at a different height within the reservoir. In such an embodiment, the controller is configured to activate only a top vibrator out of the plurality of ultrasonic vibrators based on a water level of the water in the reservoir, wherein the top vibrator is at or immediately below the water level.

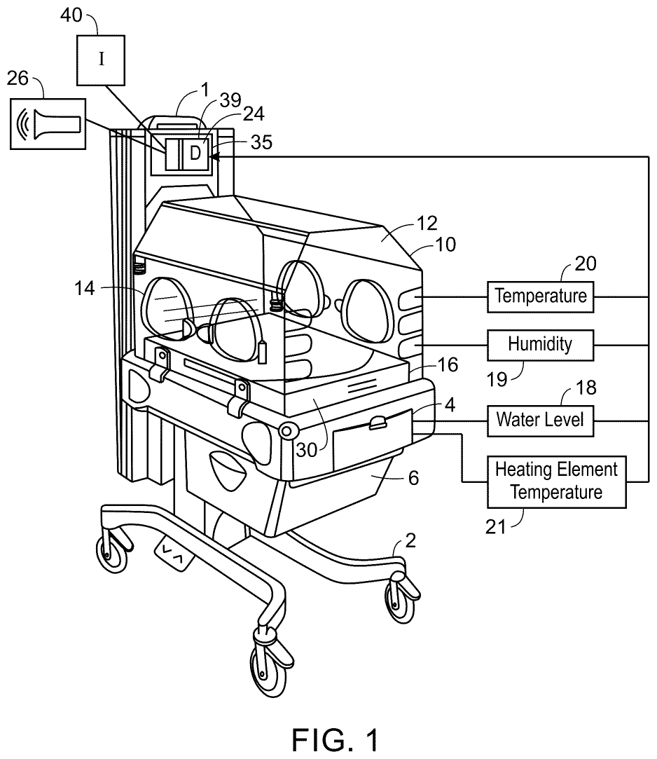

FIG. 1 depicts one embodiment of an incubator system 1 having a bed 16 supported by a base 2. In the depicted embodiment, the base 2 is on wheels to facilitate transit of an infant in the incubator system 1. The depicted incubator system 1 has a hood 10 defining a chamber 12 creating a microenvironment for housing a neonate. The hood 10 comprises a transparent housing extending above the bed 16. In the example, the hood 10 includes a plurality of portholes 14 through which a healthcare provider may access the one or more infants within the chamber 12. The depicted hood 109 comprises a single structure movable as a unit. In other implementations, sidewalls of the hoods 10 may remain about the bed 16 forming a crib, wherein the top-most portion is separable and/or removable from the sidewalls to access the one or more infants supported in the microenvironment.

The bed 16 may further include heating component(s) 30 used to control the temperature within the microenvironment of the chamber 12. For example, the chamber heater 30 may be a radiant heating or warming device that heats the air within the chamber 12 to a predefined temperature or within a predefined temperature range. In another embodiment, the air heater 30 may comprise a convective or conductive heating device or any other type of controllable heating or warming device. For example, the heater 30 may be above or below the mattress, recessed in the hood area, or elsewhere. One or more sensors may be provided to sense the air temperature and humidity within the microenvironment of the chamber 12, such as humidity sensor 19 and temperature sensor 20 configured to sense the air temperature in the microenvironment.

The incubator system 1 further includes a humidifier system 4 controllable to adjust the relative humidity within the chamber 12. The humidifier comprises a device that evaporates water, such as distilled water 7, to increase relative humidity of air within the neonatal microenvironment. The humidifier system 4 has a water reservoir 6 containing evaporant, such as distilled water, utilized for humidification of the chamber 12. The water reservoir 6 is a tank or other vessel capable of holding the evaporant. The humidifier system 4 may be located in the structure of the incubator, such as in a cavity under the mattress area, in the tower structure, or elsewhere. The humidifier system 4 is configured such that the humidified air is circulated to the chamber 12 to maintain the microenvironment therein. Although the water reservoir 6 in the illustrated embodiment is located below the bed 16, in other embodiments the water reservoir 6 may be provided at other locations. In one embodiment, the water reservoir 6 is removable from the incubator system 1 for draining and/or cleaning purposes. In other embodiments, the reservoir 6 may remain attached to the incubator system 1 and may provide other access for the purpose of draining the evaporant from the reservoir 6 and/or cleaning the reservoir 6.

A water level sensor 18 may sense the water level within the reservoir 6 and generate a water level indicator value, such as to notify a clinician that water needs to be added to the reservoir 6. In such an embodiment, the designated controller of the control system 25 may be configured to control the plurality of heating elements based on the measured water level, such as to identify and activate the top heating element closest to and below the water surface. Alternatively or additionally, the system 1 may include one or more temperature sensors 21 configured to sense temperatures of the heating elements, or temperatures of at least a subset of the heating elements, and to identify the top heating element based on the measured temperatures. Example embodiments are further described below.

The sensors 18, 19, 20, 21 provide the sensed information to the control system 35, which controls various elements within the incubator system 1. The control system 35 includes a computing system acting as one or more controllers and having at least one processor and memory capable of storing software for providing various control functions, including for controlling the humidifier to maintain a particular humidity level within the chamber 12. In the depicted embodiment, the incubator system 1 further includes a user interface 39 comprising a display 24, a speaker 26, and an input 40. Such user interface 39 elements are used to provide information to a clinician regarding the status and condition of the incubator system 1, as well as to receive control inputs from a clinician to control various aspects of the incubator system 1, including to control the environment within the chamber 12. The display 24 includes any visual output device, examples of which include a digital display screen, monitor, or the like (which may also be a touchscreen) that displays sensed values and control information about the incubator system 1 and presents visible notifications or messages to a clinician.

The display 24 may include a monitor independent of the bed portion of the incubator incorporated into some portion. In another implementation, the display 24 may comprise a screen of a portable computing or electronic device, such as a smartphone or tablet computer. Likewise, the speaker 26 may be any audio output device and may be incorporated into the bed portion of the incubator system, or may be included in a separate device, such as in the personal computing device described above. Likewise, the input 40 may be any device that facilitates user input of information, such as commands, selection, data, or settings for the incubator system 1. In one implementation, the input 40 may include a keyboard, touchpad, touchscreen, mouse, or microphone with speech recognition software, or the like. Thus, the user interface 39 may be configured to allow a user, such as a clinician, to set a humidity level for the microenvironment, and the control system 35 controls operation of the humidifier 4 to maintain the humidity at the selected humidity level. To provide just one example, the user interface may be configured to allow a clinician to set a relative humidity percentage for the microenvironment, such as a humidity level between 30% and 95%. The control system 35 then generates control instructions to control current to one or more heating elements within the humidifier system 4 to control the amount of water evaporated from the reservoir, and thereby, to control the humidity level in the microenvironment within the chamber 12.

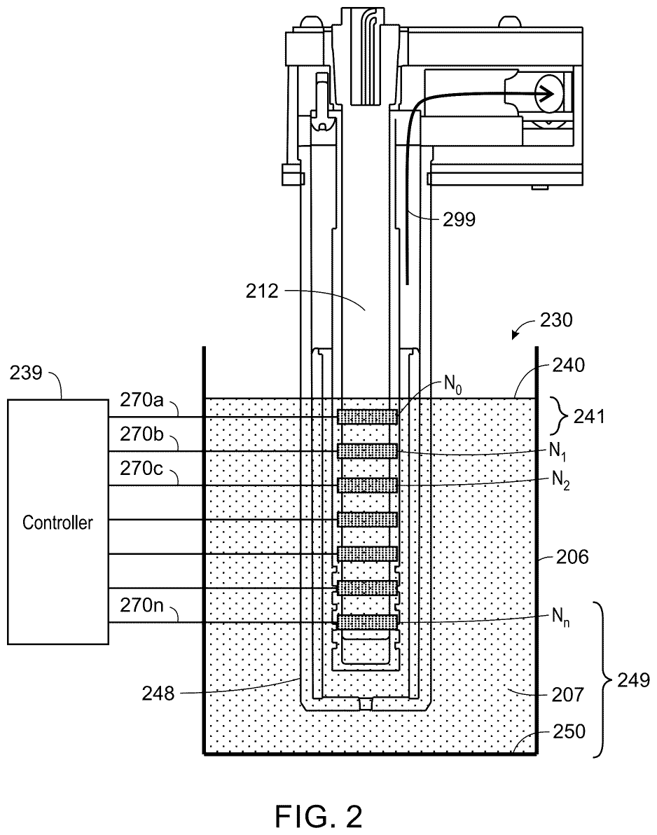

FIG. 2 represents one embodiment of a humidifier reservoir 6 and heating element configuration wherein a plurality of heating elements N₀-Nₙ are positioned at different heights in the reservoir 6 between the top side 230 and the bottom side 250 of the reservoir 206. The controller 239 controls the heating elements N₀-Nₙ individually to heat the surface region of the water 207 to a higher temperature than a bottom region 249 of the water 207 that is closer to the bottom side 250 of the reservoir 206. Thus, the water at and near the water level 240 is the hottest and the water in the bottom region 249 adjacent to the bottom side 250 of the reservoir 206 is the coldest. The surface region 241 is immediately below the water level 240, which is the top surface of the water 207 in the reservoir, and occupies the top surface and the area immediately below that is heated to cause evaporation. For example, the surface region 241 is heated by a top heating element, which is the heating element closest to and immediately below the water level 240. Thus, minimal or no bubbling is generated, and bubble collapse does not occur, thus noise is not generated.

In some embodiments, only the top heating element out of the plurality of heating elements N₀-Nₙ is powered at any given time. The top heating element (which is N₀ in the scenario shown in FIG. 2) is the highest heating element that is below the water level 240, which is in the surface region 241 and the location of the top heating element and/or the spacing of the plurality of heating elements may define the depth of the surface region 241. In other embodiments, one or more heating elements immediately below the top heating element may also be powered, such as to a lesser temperature than the top heating element. Thus, while a maximum power amount is provided to the top heating element, a lesser power amount that is less than the maximum power amount is provided to the subset of heating elements below the top heating element.

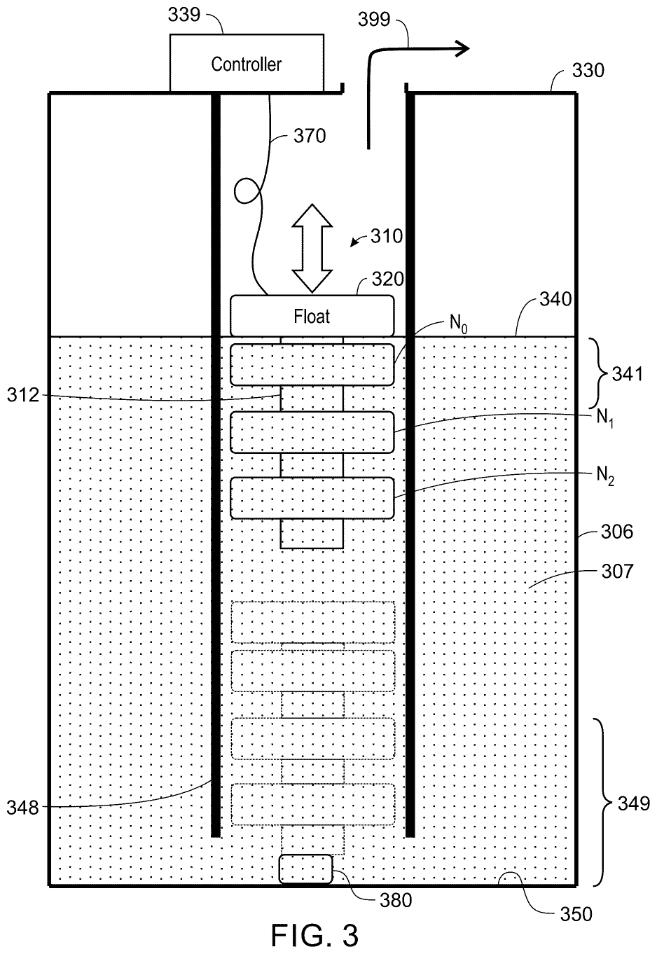

As the water level 240 in the reservoir 206 moves up and down, the heating by the plurality of heating elements N₀-Nₙ moves up and down accordingly such that the maximal heating is maintained in the surface region 241. Heating water to the boiling point in the bottom region 249, or the water that is otherwise well below the surface region 241, is sufficiently avoided so as not to cause gas bubbles to develop and rise. Thus, in some embodiments, the water in the lower layers may be heated, but not to the point where bubbles are formed due to boiling. In embodiments with fixed heating elements, like that shown in FIG. 2, the power to the plurality heating elements N0-Nₙ is adjusted to align with the water level such that the heat generated is concentrated in and near the surface region 241. In other embodiments, like that exemplified in FIG. 3, the plurality of heating elements N₀-Nₙ is on a floating heater system 310 that is suspended from the top surface of the water such that the plurality of heating elements N₀-Nₙ moves up and down with and is maintained at a consistent depth below the water level.

Returning to FIG. 2, the controller 239 may be configured to identify the top heating element and control power to the plurality of heating elements N₀-Nₙ accordingly. Identification of the top heating element may be based on a sensed water level, such as by a water level sensor, or may be based on temperature sensing of the heating elements N₀-Nₙ. Where a water level sensor is used, the sensor may be configured to sense the water level 240 by any means suitable. For example, the water level sensor may be a resistive sensor, a float sensor, a time-of-flight sensor, or any other sensing means known to be suitable for sensing water level. In such an embodiment, the controller 239 is configured to receive the sensed water level and may be configured to execute logic to identify the top heating element based on the sensed water level. For example, the controller 239 may store and access a table or other logic structure by which water levels are associated with a top heating element.

The controller 239 is configured to control power delivered to each of the plurality of heating elements N₀-N_{n.} Each heating element N₀-Nₙ may be connected to a conductive wire 270a-270n and the controller 239 is configured to individually control power delivered from a power source to each of the plurality of heating elements N₀-Nₙ through the respective wires 270a-270n. The arrangement of the wires 270a-270n in the figures is for illustrative purposes and visual clarity. The wires 270a-270n may be embedded in the structure housing the plurality of heating elements N₀-Nₙ, such as through the pillar 212 on which the heating elements N₀-Nₙ are mounted. Referencing the depicted example, the top heating element N₀ because it is immediately below the water level 240. The top element N₀ would be heated to a maximum level by delivering a maximum current to that heater via the respective wire 270a. At least a subset of the heating elements below the top element N₀ would be heated to a lesser degree, or not at all. Thus, less or no current would be delivered to heating elements N₁ and N₂ via wires 270b and 270c. As the water level 240 moves down, the top element designation moves down accordingly and current to the plurality of heating elements N₀-Nₙ is also adjusted accordingly.

Heating the water causes evaporation of the water 207, which travels out of the reservoir, such as out the top side 230 along pathway 299. In some embodiments, the heater plurality of heating elements N₀-Nₙ may be positioned within a heating chamber defined by a divider 248. The divider 248 is configured to let water 207 flow in, such as from the bottom, such that the water level in the chamber is the same as the water level 240 within the rest of the reservoir. In some embodiments, the chamber divider 248 is configured to restrict the flow of water in and out of the chamber to reduce the amount of water exposed to the heating elements and thus provide more efficient evaporation. In some embodiments, the divider 248 is comprised of a thermally resistant material, such as plastic, to maintain the heat from the heating elements N₀-Nₙ within the water maintained in the heating chamber. The divider 248 may be positioned anywhere within the reservoir 6 so as to define the heating chamber at any location within the reservoir 6, such as vertically defined within a center portion or an edge portion of the reservoir 6. The divider 248 may extend upward out of the reservoir to guide the humidified air flow along pathway 299 out of the reservoir and to the microenvironment of the incubator.

FIG. 3 depicts an embodiment wherein the plurality of heating elements N₀-Nₙ are on a floating heater system 310 that floats on the water 307 within the reservoir 306. The reservoir has a top side 330 and a bottom side 350, and the floating heater moves vertically within the reservoir 306 as the water level 340 moves up and down. The floating heater system 310 comprises a float 320 configured to provide buoyancy and to suspend the heating elements N₀-N₂ below the water level 340. The depicted embodiment includes three heating elements N₀-N₂; however, in other embodiments, the floating heater system 310 may include any number of two or more heating elements maintained at different heights below the surface water level 340.

The float 320 may be hollow, for example, a hollow structure built of lightweight plastic that is configured to withstand water exposure and heat. The plurality of heating elements N₀-N₂ are on a structure that extends downward from the float 320, such as a pillar 312 on which the plurality heating elements N₀-Nₙ mounted so as to be maintained at different heights below the surface water level 340. The top heating element N₀ is always closest to and immediately below the water level 340. One or more additional heating elements N₁-N2 (to Nₙ) are below the top heating element N₀. The heating elements N₀-Nₙ may be, for example, resistive heating elements embedded in one or more materials configured to transmit the heat to the water 307, such as a ceramic or other conductive composite material. In one embodiment, each of the heating elements N₀-Nₙ may be a nichrome wire surrounded by an electrically insulating but thermally conducting material such as magnesium oxide, held in place inside an outer thermally conducting shell that could be made of alloys of aluminum and/or other such materials. While the depicted embodiments show discrete disc like structures for the heaters N₀-Nₙ, other formations for the heating elements are possible and within the scope of the present disclosure. For example, the heating elements N0-Nₙ may be housed in one vertical tube wherein the heating elements N₀-Nₙ are formed as internally segmented zones.

The top heating element N₀ is powered the most, and thus provided a maximum power amount to heat the surface region 341. The subset of heating elements N₁-N2 (to Nₙ) below the top heating element are powered to a lesser amount such that they generate less heat than the top heating element N₀. Thus, the water below the surface region is cooler than the water in the surface region 341. The water in the bottom region 349 closer to the bottom side 350 of the reservoir 306 is the coldest since no heating elements reach the bottom region 349 until the water has reached a low water threshold.

The heating elements may have separate wires, similar to that represented in FIG. 2, and such wires may run through the structure of the pillar 312 and the float 320. A set of wires 370 connects the controller 339 and power source to the plurality of heating elements N₀-N₂ such that power is provided to the heating elements N₀-N₂. In the depicted embodiment, wires 370 extend from the controller 339 (which here is shown to include power control from a power source (not shown)) connecting to the heating circuit in the floating heater system 310. The wires 370 are provided with sufficient slack such that the floating heater system 310 can move freely within the range of surface levels between a maximum fill level and a minimum fill level. In the figures, the wires 370 are shown as having slack contained within the heating chamber defined by divider 348. The purpose and construction of the divider 348 is described with respect to FIG. 2, including to guide the humidified air along pathway 399 out of the reservoir 306 to the microenvironment. In an embodiment with a floating heater system 310, the divider may serve an added purpose of maintaining the floating heater system 310 in place within the reservoir 306 so that it efficiently heats the water 307. In other embodiments, the wires 370 may travel within a guide configured such that the floating heater 310 slides down the guide as the water level 340 decreases. Where one is provided, the guide may have one or more openings, or slots, running the length of the guide (or at least between the maximum fill level and minimum fill level) such that the wires connect with the heating elements of the floating heater system 310.

The heating elements N₀-N₂ move down in parallel with the water level 340 until the water level 340 becomes low. A low water level limit switch 380 may be configured to sense when the floating heater system 310 has reached a low point at or near the bottom side 350 of the reservoir 6. Alternatively, a different sensing arrangement may be configured to sense the position of the floating heater system 310 and/or the water level 440 to determine that the floating heater 310 has reached its lowest position. At that point, the controller 339 may be configured to generate a refill alert instructing that the reservoir be refilled.

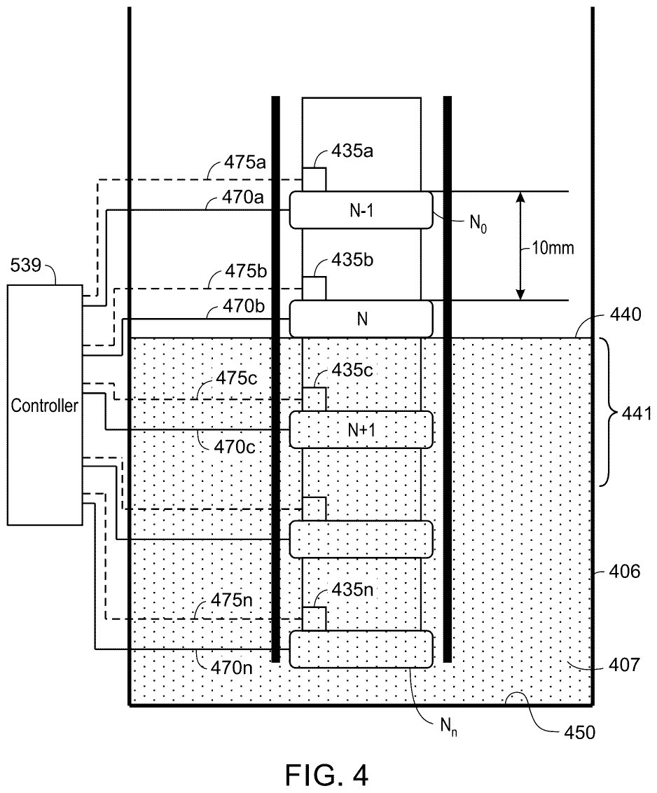

FIG. 4 depicts one embodiment of a humidifier system with stationary heating elements N₀ Nₙ and a plurality of temperature sensors 435a-435n configured to sense the temperature of the plurality of heating elements N0-Nₙ. The temperature sensors 435a-435n may be, for example, thermistors configured to sense the temperature of a respective heating element N0-Nₙ. For instance, the temperature sensors 435a-435n may be thermistors that are part of a voltage divider network, where the voltage across the thermistor indicates a temperature, which is an analog signal that can be fed to the controller. Alternatively, the temperature sensors 435a-435n may be a different type of integrated circuit or other sensor structure configured to sense or otherwise identify the temperature of each, or at least a subset of, the plurality of heating elements N0-Nₙ.

The measured temperature is communicated to the controller 539 via the communication link(s) 475a-475n. In some embodiments, each sensor 435a-435n may have a dedicated communication link 475a-475n, as is represented in the figure. In other embodiments, all or a subset of the sensors 435a-435n may communicate the measured temperature on a shared communication link, such as a serial bus or a wireless communication means. The plurality of plurality of heating elements N0-Nₙ are then controlled accordingly by the controller 539 to deliver maximum power via wires 470a-470c to generate maximum temperature at the top heating element and, in various embodiments, lower or no heat output to the subset of heating elements N0-Nₙ that are below the top heating element (shown in FIG. 4 as N+1). Thereby, the surface region 441 of water 407 within the reservoir 406 is heated more, and maintained at a higher temperature during operation of the humidifier, than the water closer to the bottom side 450 of the reservoir 406. The heating elements above the top element N+1, shown in FIG. 4 as heating elements N and N−1 may be turned off and not powered since they are not immersed in water. In other embodiments, the power of those heating elements above the top element may be powered to a threshold output below the maximum power, such as to heat the air above the water level 440 but without overheating.

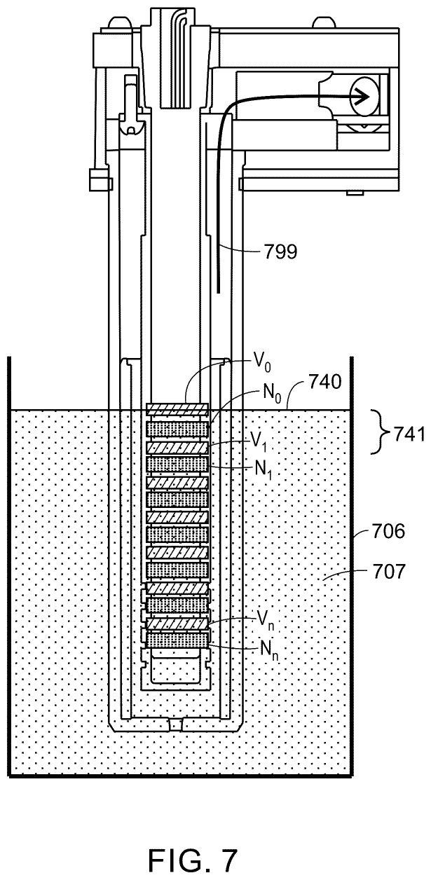

The plurality of heating elements N₀-Nₙ are spaced sufficiently close together such that the top element (shown in FIG. 4 as N+1) is always within a maximum depth below the water level 440 permitted for the surface region 441. The maximum depth permitted for the surface region 441 is configured such that bubbling is minimized and bubble collapse does not occur. For example, the plurality of heating elements N₀-Nₙ may be positioned every 10 mm vertically at different heights within the reservoir 406. In other embodiments, the spacing may be more or less, and may depend on the size and maximum operating temperature of the heating elements. In some embodiments, such as that shown in FIG. 7, one or more ultrasonic vibrators may be configured to aerosolize the water to generate humidity. In such an embodiment, the heating elements N₀-Nₙ may be spaced further apart, such as where the water is maintained below boiling and/or where ultrasonic vibrators are positioned between the heating elements N₀-Nₙ. Embodiments are further described below with respect to FIG. 7.

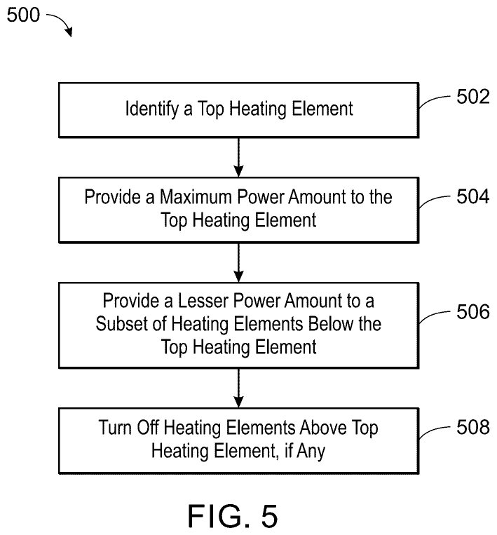

FIG. 5 depicts a method executed by the controller for controlling a humidifier system of a neonatal incubator having a plurality of heating elements at different heights according to the present disclosure. At step 502, logic is executed to identify a top heating element. In various examples described herein, the top heating element may be identified based on the measured water level and/or based on the measured temperature of the heating elements. A maximum power amount is provided to the top heating element at step 504 to heat the surface region of the water at and below the water level to a higher temperature than the water closer to the bottom side of the reservoir. A lesser power amount is provided to the remaining heating elements, which may be provided some power or not powered at all. For example, the heating elements below the top heating element may be powered to a lesser power among at step 506 such that they produce some heat, but less than the top heating element and not enough that they cause the surrounding water below the surface region to boil. The heating elements above the top heating element, if any, may not be powered at all and thus turned off at step 508.

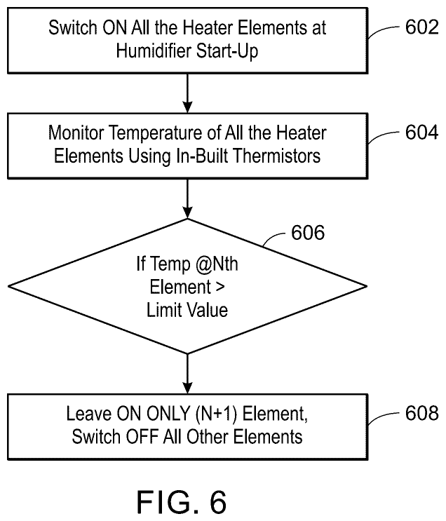

In embodiments with temperature sensors 435a-435n configured to sense temperature of the plurality of heating elements N₀-Nₙ, the controller may be configured to execute logic to identify the top heating element based on the measured temperatures. FIG. 6 illustrates one embodiment of logic for identifying the top heating element based on measured temperature. Upon startup of the humidifier, such as upon activation of the humidifier by the controller based on a measured humidity and/or measured temperature within the microenvironment, steps may be executed to identify a top heating element. In the depicted example, all of the plurality of heating elements are switched on at step 602, and thus powered by the controller. For example, a predefined power amount (which may be significantly lower than a maximum power amount for the heaters) is delivered to each of the plurality of heating elements. The temperature of all of the plurality of heating elements is measured at step 602, such as by temperature sensors shown and described in the embodiment in FIG. 4. The temperature is then correlated to the presence or absence of the water around the respective heating element. The measured temperatures, such as the temperature magnitudes or the rate of temperature change, are each compared to a threshold temperature at step 606 to determine which heating elements exceed a threshold temperature or threshold rate of change. In one embodiment, the threshold temperature may be determined based on the measured temperatures, such as based on a temperature jump (difference in temperature differentials between adjacent heating elements along the vertical arrangement of heaters) or difference in rate of temperate rise or spread between the temperature measurements of the heating elements that are submersed in the water versus those that are at least partially above the water surface. The heating elements that are at least partially or fully above the water surface will heat up more than the submersed heating elements due to the lower conductivity of air compared to water. The amount and location of that temperature jump may be utilized to identify the heaters that are not immersed in water (e.g., elements N and N−1 in FIG. 4). The top heating element is identified as the highest heating element that is immersed, and thus below the threshold. Once the top heating element is identified, the remaining heating elements may be turned off at step 608 such that only the top heating element (element N+1 in FIG. 4) is operated to heat the surface region. In other embodiments, some or all of the other heating elements may be operated at a lower power level, as is described herein.

Additional steps may then be executed to identify when the water level has fallen below the top heating element. For example, the temperature of the top heating element may be continually monitored to detect when it exceeds the temperature threshold indicating that it is no longer submerged. At that point, the next heating element below the current top heating element is energized and becomes the top heating element.

The system may be configured to include one or more ultrasonic vibrators configured to aerosolize the water heated by the heating system comprising the plurality of heaters at different heights in the reservoir. The plurality of heating elements is controlled to maintain the surface region of the water within a predetermined temperature range, as is described above, which may include measuring a temperature of the heating element and/or the water in the surface region and powering at least the top heating element accordingly. In some embodiments, particularly where the system includes ultrasonic vibrators, the water may be heated to a temperature that is below boiling and the ultrasonic vibrator(s) may be operated to aerosolize the heated water. Heating the water has the benefit of killing bacteria and other organisms to avoid transmission of infection to an infant maintained in the microenvironment. In other embodiments, the water may be heated to boiling temperature in combination with the ultrasonic vibration.

FIG. 7 depicts an exemplary humidifier system comprising a plurality of ultrasonic vibrators V₀-Vₙ positioned at different heights within the reservoir. Here, the ultrasonic vibrators V₀-Vₙ are positioned between the heating elements N₀-Nₙ and operated following the top heater determination logic, various embodiments of which are described herein. Namely, a top heating element (here, N₀) and an associated top ultrasonic vibrator (here, V₀) in the surface region 741 are operated to generate humidified air that is passed along pathway 799 to humidify the microenvironment. The top heating element (here, N₀) is below the water level 740 and remains completely submerged. Once the water level 740 drops such that the top ultrasonic vibrating elements V₀ is completely above the water surface (completely out of the water 707) and/or the top heating element N₀ is no longer totally submerged, the controller will designate the next heating element/vibrator pair below as the new top elements. This determination may be based on sensed water level or temperature sensing, as is exemplified and described above.

The top ultrasonic vibrator (here, V₀) is one of the plurality of vibrator elements V₀-Vₙ associated with the heating element N₀-Nₙ designated as the top heating element, which may be based on sensed water level and/or sensed temperature information, as is described above. In the depicted example, each heating element N₀-Nₙ is associated with a respective ultrasonic vibrating element V₀-Vₙ, where the ultrasonic vibrating element V₀-Vₙ is immediately above the heating element N₀-Nₙ with which it is associated. In other embodiments, the ultrasonic vibrating element V₀-Vₙ is immediately below the heating element N₀-Nₙ with which it is associated.

Alternatively, the ultrasonic vibrating element may be on a floating element configured to float on the surface of the water level 740, such as one or two vibrating elements suspended on or immediately below the water surface by a float. In such an embodiment, the plurality of heating elements may be on a stationary heater system such as shown in FIGS. 1, 4, or 7, or may be on a floating heating system like that shown in FIG. 3. For example, the floating ultrasonic element could be a ring-like structure that moves (floats) up or down around the cylindrical heater housing inside the partition 248, and always acts on the surface of the water.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A humidifier system for humidifying a microenvironment in a neonatal incubator, the
humidifier system comprising:
a reservoir configured to hold water to be evaporated for humidifying the microenvironment, the reservoir having a top side and a bottom side;
a plurality of heating elements, each heating element positioned at a different height between the top side and the bottom side of the reservoir;
a controller configured to control power to each of the plurality of heating elements to heat a surface region of the water to a higher temperature than a bottom region of the water closer to the bottom side of the reservoir.

2. The system of claim 1, wherein the controller is further configured to turn off power to individual heating elements of the plurality of heating elements as a water level of the water decreases.

3. The system of claim 1, wherein the plurality of heating elements includes at least a first heating element and a second heating element, wherein the first heating element is above the second heating element in the reservoir, and wherein the controller is further configured to:
determine that the first heating element is above a water level of the water in the reservoir; and
turn off power to the first heating element and increase power to the second heating element.

4. The system of claim 1, wherein the system further comprises a water level sensor configured to measure a water level of the water in the reservoir, and wherein the controller is configured to control the plurality of heating elements based on the measured water level.

5. The system of claim 1, wherein the system further comprises a plurality of temperature sensors, wherein each temperature sensor is configured to measure a temperature of a respective one of the plurality of heating elements, and wherein the controller is configured to control the plurality of heating elements based on the measured temperatures of the plurality of heating elements.

6. The system of claim 1, wherein the controller is further configured to provide more power to a top heating element than the other heating elements in the plurality of heating elements, wherein the top heating element is the heating element out of the plurality of heating elements that is at or immediately below a water level of the water in the reservoir.

7. The system of claim 6, wherein the controller is configured to:
identify the top heating element out of the plurality of heating elements, wherein the top heating element is the highest heating element that is below a water level of the water in the reservoir;
provide a maximum power amount to the top heating element; and
provide a lesser power amount to each of a subset of heating elements below the top heating element, wherein the lesser power amount is less than the maximum power amount.

8. The system of claim 7, wherein the controller is configured to identify a new top heating element out of the plurality of heating elements as the water level decreases such that the new top heating element remains completely submerged and within the surface region.

9. The system of claim 6, wherein the plurality of heating elements are in a floating heater system configured to float such that at least the top heating element is maintained within the surface region and at least one lower heating element is maintained below the surface region, wherein the controller is configured to:
provide a maximum power amount to the top heating element; and
provide a lesser power amount to each of the at least one lower heating element, wherein the lesser power amount is less than the maximum power amount such that the water in the surface region is heated more than the water below the surface region.

10. The system of claim 1, further comprising at least one ultrasonic vibrator positioned in the surface region and configured to aerosolize the water in the surface region that is heated by the at least one heating element.

11. The system of claim 10, wherein the system further comprises a plurality of ultrasonic vibrators each positioned at a different height within the reservoir;
wherein the controller is further configured to activate only a top vibrator out of the plurality of ultrasonic vibrators based on a water level of the water in the reservoir, wherein the top vibrator is at or immediately below the water level.

12. The system of claim 11, wherein the controller is configured to control the plurality of heating elements to maintain the surface region of the water within a predetermined temperature range that is below boiling.

13. The system of claim 10, wherein the at least one ultrasonic vibrator is in a floating element configured to maintain the at least one ultrasonic vibrator in the surface region of the water as the water level changes.

14. A neonatal incubator comprising:
a bed configured to support a neonate;
a hood above the bed configured to encapsulate a microenvironment around the neonate;
a humidifier system configured to humidify the microenvironment, the humidifier system having:
a reservoir configured to hold water to be evaporated for humidifying the microenvironment, the reservoir having a top side and a bottom side;
a plurality of heating elements, each heating element positioned at a different height within the reservoir; and
a controller configured to control power to each of the plurality of heating elements based on a water level of the water in the reservoir and the measured humidity so as to heat a surface region of the water to a higher temperature than a bottom region of the water closer to the bottom side of the reservoir.

15. The neonatal incubator of claim 14, wherein the plurality of heating elements includes at least a first heating element and a second heating element, wherein the first heating element is above the second heating element in the reservoir, and wherein the controller is further configured to:
determine that the first heating element is above the water level; and
turn off power to the first heating element and increase power to the second heating element.

16. The neonatal incubator of claim 14, wherein the humidifier system further comprises a plurality of temperature sensors, wherein each temperature sensor is configured to measure a temperature of a respective one of the plurality of heating elements, and wherein the controller is configured to control the plurality of heating elements based on the measured temperatures of the plurality of heating elements.

17. The neonatal incubator of claim 14, wherein the plurality of heating elements are in a floating heater system configured to float in the reservoir such that at least a top heating element is maintained within the surface region and at least one lower heating element is maintained below the surface region, wherein the controller is configured to:
provide a maximum power amount to the top heating element; and
provide a lesser power amount to each of the at least one lower heating element, wherein the lesser power amount is less than the maximum power amount such that the water in the surface region is heated more than the water below the surface region.

18. The neonatal incubator of claim 14, wherein the controller is configured to:
identify a top heating element out of the plurality of heating elements, wherein the top heating element is the highest heating element that is below a water level of the water in the reservoir;
provide a maximum power amount to the top heating element; and
provide a lesser power amount to each of a subset of heating elements below the top heating element, wherein the lesser power amount is less than the maximum power amount.

19. The neonatal incubator of claim 14, wherein the system further comprises a plurality of ultrasonic vibrators each positioned at a different height within the reservoir;
wherein the controller is further configured to:
control power to the plurality of heating elements to maintain the surface region of the water within a predetermined temperature range that is below boiling; and
activate only a top vibrator out of the plurality of ultrasonic vibrators based on the water level, wherein the top vibrator is at or immediately below the water level.

20. A method of controlling a humidifier system in a neonatal incubator, the humidifier system comprising a reservoir configured to hold water to be evaporated for humidifying the microenvironment and a plurality of heating elements, each heating element positioned at a different height between a top side and a bottom side of the reservoir, the method comprising:
identifying a top heating element out of the plurality of heating elements, wherein the top heating element is the highest heating element that is below a water level of the water in the reservoir;
providing a maximum power amount to the top heating element; and
providing a lesser power amount to each of a subset of heating elements below the top heating element, wherein the lesser power amount is less than the maximum power amount.
